(19) United States
(12) Patent Application Publication
(10) Pub. No.: US 2026/0265185 A1
(43) Pub. Date: Sep. 10, 2026

(54) HIGH PURITY NITRIC OXIDE RELEASING COMPOUNDS AND METHODS OF PREPARING SAME

(71) Applicant: KNOW Bio, LLC, Morrisville, NC (US)

(72) Inventors: Mona Jasmine R. Ahonen, Durham, NC (US); John Kelly Simons, Cary, NC (US); Thomas Goldthwaite Coleman, Pittsboro, NC (US)

(73) Assignee: KNOW Bio, LLC, Morrisville, NC (US)

(21) Appl. No.: 19/162,899

(22) PCT Filed: Mar. 5, 2024
(86) PCT No.: PCT/US2024/018469

§ 371 Date: Sep. 5, 2025

Related U.S. Application Data
(60) Provisional application No. 63/488,775, filed on Mar. 7, 2023.

(51) Int. Cl. C07C 291/02

(52) U.S. Cl. CPC C07C 291/02; C07B 2200/13

(57) ABSTRACT

Provided herein is a method of manufacturing a high purity nitric oxide releasing compound product preparation. Also described herein is a high purity nitric oxide releasing compound product preparation prepared according to the manufacturing method. A high purity nitric oxide releasing compound product, having a purity of at least 97 area % as measured by high pressure liquid chromatography and a residual solvent level of less than 1.5% is further described herein.

### CROSS-REFERENCE TO PRIORITY APPLICATION

This application claims priority to U.S. Provisional Application No. 63/488,775, filed Mar. 7, 2023, which is incorporated herein by reference in its entirety.

### FIELD

The present disclosure relates to a method of manufacturing a high purity nitric oxide releasing compound product preparation. The present disclosure also relates to high purity nitric oxide releasing compound products, having low levels of impurities and/or residual solvent.

### BACKGROUND

Manufacturing processes can have a significant impact on the suitability of a drug product for patient administration. For example, compound byproducts and residual solvent can remain in the final compound form, thus deeming the compound unsuitable for further use. Methods of minimizing drug related process impurities and residual solvents are important in a drug manufacturing process.

### SUMMARY

Provided herein is a method of manufacturing a high purity nitric oxide releasing compound product preparation, comprising preparing in a vessel a reaction mixture comprising a hydroxide or a hydroxide donor and one or more reaction mixture solvents; introducing nitric oxide into the vessel under an inert atmosphere to obtain a suspension comprising a nitric oxide releasing compound; filtering the suspension to obtain a nitric oxide releasing compound solid; dissolving the nitric oxide releasing compound solid in an aqueous solution to form a dissolved nitric oxide releasing compound solution; recrystallizing the nitric oxide releasing compound solid from the dissolved nitric oxide releasing compound solution to form a recrystallized nitric oxide releasing compound product, wherein the recrystallizing comprises a step of adding methanol to the dissolved nitric oxide releasing compound solution; filtering the recrystallized nitric oxide releasing compound product and washing the filtered recrystallized nitric oxide releasing compound product with a mixture of washing solvents to form a washed nitric oxide releasing compound product, wherein the mixture of washing solvents comprises methanol and one or more additional alcohols; drying the washed nitric oxide releasing compound product to form a dried nitric oxide releasing compound product; and dissolving the dried nitric oxide releasing compound product in water to form the high purity nitric oxide releasing compound product preparation.

Optionally, the high purity nitric oxide releasing compound product preparation has a purity of at least 97 area % or at least 98 area % as measured by high pressure liquid chromatography. Optionally, the high purity nitric oxide releasing compound product preparation has an impurity level of less than 1 area % or less than 0.5 area % as measured by high pressure liquid chromatography. In some cases, the high purity nitric oxide releasing compound product preparation has a residual solvent level of less than 1.5% w/w (e.g., less than 1.0% w/w).

The reaction mixture can optionally comprise a hydroxide selected from the group consisting of sodium hydroxide, potassium hydroxide, lithium hydroxide, calcium hydroxide, magnesium hydroxide, or ammonium hydroxide. Optionally, the reaction mixture can comprise a hydroxide donor selected from the group consisting of sodium methoxide, lithium methoxide, potassium methoxide, calcium methoxide, magnesium methoxide, or ammonium methoxide.

The one or more reaction mixture solvents can comprise an alcohol, such as an alcohol selected from the group consisting of methanol, ethanol, n-propanol, isopropanol, or isobutanol. Optionally, the one or more reaction mixture solvents can comprise a ketone, such as acetone, dihydroxyacetone, and dimethyl malonate. The aqueous solution can optionally comprise water.

In some cases, the recrystallizing step comprises adding a nitric oxide releasing compound seed during the step of adding methanol to the dissolved nitric oxide releasing compound solution. Optionally, the step of adding the nitric oxide releasing compound seed is initiated after a concentration of methanol in the dissolved nitric oxide releasing compound solution reaches at least 15% (v/v) or at least 20% (v/v). The nitric oxide releasing compound seed can be added to the dissolved nitric oxide releasing compound solution as a powder or as a suspension in a solvent (e.g., methanol and water).

Optionally, the mixture of washing solvents comprises methanol and a C₂-C₂₀ alcohol, wherein the C₂-C₂₀, alcohol is miscible with methanol. In some cases, the C₂-C₂₀ alcohol is selected from the group consisting of ethanol, propanol, isopropanol, butanol, or isobutanol. Optionally, the mixture of washing solvents comprises a ratio of methanol to C₂-C₂₀ alcohol of 1:1 to 1:10.

The drying can be performed under vacuum. Optionally, the dried nitric oxide releasing compound product is a crystalline form.

In some cases, the nitric oxide releasing compound comprises a compound having at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation. Optionally, the compound has the following structure:

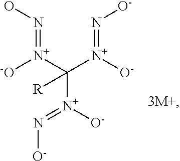

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H; a, b, c, d, e, and f are each independently selected from an integer of 0, 1, 2, 3, 4, 5, 6, 7, 8, 9, or 10; and M+ is a pharmaceutically-acceptable cation, wherein a ratio of the compound to the cation is such that the overall net charge of the compound is neutral.

In some examples, the cation is selected from the group consisting of sodium, potassium, lithium, calcium, magnesium, ammonium, and substituted ammonium. Optionally, the compound has the following structure:

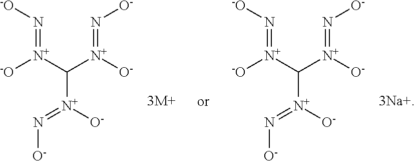

Further described herein is a high purity nitric oxide releasing compound product preparation prepared according to the method as described herein. Optionally, the high purity nitric oxide releasing compound product preparation comprises a sealed container.

Also described herein is a high purity nitric oxide releasing compound product, comprising a compound having at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation, wherein the high purity nitric oxide releasing compound product has a purity of at least 97 area % as measured by high pressure liquid chromatography; and wherein the high purity nitric oxide releasing compound product has a residual solvent level of less than 1.5% w/w.

Optionally, the compound has the following structure:

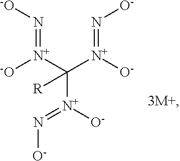

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H; a, b, c, d, e, and f are each independently selected from an integer of 0, 1, 2, 3, 4, 5, 6, 7, 8, 9, or 10; and M+ is a pharmaceutically-acceptable cation, wherein a ratio of the compound to the cation is such that the overall net charge of the compound is neutral. In some examples, the cation is selected from the group consisting of sodium, potassium, lithium, calcium, magnesium, ammonium, and substituted ammonium. Optionally, the compound has the following structure:

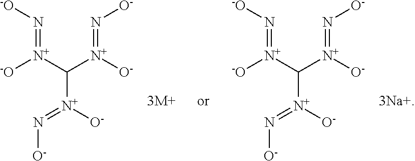

In some cases, the purity of the high purity nitric oxide releasing compound product is at least 98 area % as measured by high pressure liquid chromatography. Optionally, the high purity nitric oxide releasing compound product has an impurity level of less than 1 area % (e.g., less than 0.5 area %) as measured by high pressure liquid chromatography. Optionally, the residual solvent level of the high purity nitric oxide releasing compound product is less than 1.0% w/w. The high purity nitric oxide releasing compound product is optionally crystalline.

The details of one or more embodiments are forth in the drawings and the description below. Other features, objects, and advantages will be apparent from the description and drawings, and from the claims.

### DESCRIPTION OF THE DRAWINGS

FIG. 1 is a schematic depicting the design of reverse addition reprecipitation studies with MD3.

FIG. 2A shows the effect of stir speed on % MD3, MD2, and MeOH for the reverse addition reprecipitation study samples. FIG. 2B shows the effect of addition rate on % MD3, MD2, and MeOH for the reverse addition reprecipitation study samples.

FIGS. 3A, 3B, and 3C show the effect of wash step on % MD3 (FIG. 3A), MD2 (FIG. 3B), and MeOH (FIG. 3C) MeOH for the reverse addition reprecipitation study samples.

FIG. 4 is a schematic depicting the design of normal addition reprecipitation studies with MD3.

FIG. 5 is a photograph of crystalline MD3 particles.

FIG. 6 depicts the MD3 crystal structure.

FIG. 7 is a graph of the MD3 solubility curve in a MeOH:water solvent system.

FIGS. 8A and 8B contain high pressure liquid chromatography chromatograms demonstrating the purity level of a recrystallized MD3 sample.

### DETAILED DESCRIPTION

Described herein are high purity nitric oxide releasing compound products and product preparations (e.g., sealed, storage appropriate containers including the high purity nitric oxide releasing compound product). The high purity nitric oxide releasing compound products are prepared according to the methods described herein, which are suitable for large-scale (process scale) manufacturing of the products. The methods described herein result in drug products suitable for administration to a subject and include minimal process impurities and residual solvents.

Notably, the methods described herein are directed to the synthesis of N-diazeniumdiolates with pharmaceutical-grade purity (e.g., 98% or greater purity). Of note, the use of N-diazeniumdiolates has been studied and utilized extensively to produce NO spontaneously with first order release kinetics under physiologic conditions. Although the concept of using N-diazeniumdiolate NO donors as prodrugs that release NO upon exposure to aqueous environments dates to the early 2000s, no such C-diazeniumdiolates have been commercialized for any clinical indication due to the manufacturing and handling (including stability) challenges that require significant investment.

The methods described herein, related to both the manufacture and formulation of MD3 into the drug product, address and avoid the difficulties of this compound. Using the methods described herein, the N-diazeniumdiolates are stable for extended periods (for example, at least 18 months at −20° C.) when stored as a liquid under appropriate conditions. Packaging diazeniumdiolates in an aqueous solution is a surprising feature of the present method, and contrary to common understanding in the art, because water is the trigger for decomposition of the prodrug and subsequent NO-release. However, a careful balance of conditions and steps, as described herein, unexpectedly maintains stability of the N-diazeniumdiolates and still exerts the desired pharmacologic properties of NO until the final formulation is delivered into the lungs of a patient.

### High Purity Nitric Oxide Releasing Compound Products and Product Preparations

High purity nitric oxide releasing compound products are described herein. A nitric oxide releasing compound as described herein can have at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation. In some cases, the nitric oxide releasing compound includes three diazeniumdiolate groups on one carbon atom.

Although various NO donors (e.g., diazeniumdiolates, S-nitrosothiols, metal nitrosyls, organic nitrates) are known to provide for controlled exogenous NO release, the diazeniumdiolate functional group (NONOate) in the compounds disclosed herein are attractive because of their good stability and facile storage, and because they spontaneously undergo proton-triggered dissociation under physiological conditions to regenerate nitric oxide. Certain compounds include two diazeniumdiolate groups on one carbon atom, each having a charge and each with an associated pharmaceutically-acceptable cation to balance the charge on the diazeniumdiolate groups. The compounds are small molecules (having a molecular weight of 500 g/mol or less, without the cation, as further described below) that release nitric oxide (NO) and exhibit antimicrobial characteristics.

Optionally, the compound has the following structure, as represented by Formula I:

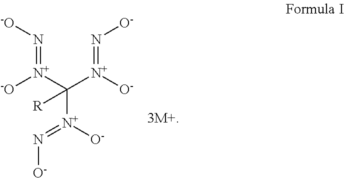

In Formula I, R is hydrogen, deuterium, C₁₋₁₂ alkyl, aryl, heteroaryl, alkylaryl, arylalkyl, or carbonyl. Optionally R is substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H, wherein a, b, c, d, e, and f are each independently selected from an integer of 0, 1, 2, 3, 4, 5, 6, 7, 8, 9, or 10.

Additionally in Formula I, M+ is a cation. For example, M+ can be a pharmaceutically acceptable cation. Optionally, the cation is selected from the group consisting of sodium, potassium, lithium, calcium, magnesium, and quaternary ammonium salts (e.g., ammonium or substituted ammonium).

In these compositions, a ratio of the compound to the cation is such that the overall net charge of the compound is neutral. In cases where M+ is a cation with a valence other than one, a ratio of the compound to the cation is such that the total positive charge equals the total negative charge. By way of example, for a compound having a total charge of negative three, and a cation with a total charge of positive one, there would be one compound and three cations.

For example, the compound can be represented by Structure I-A, as shown below:

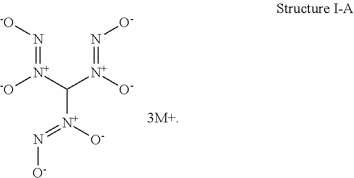

In Structure I-A, M+ is defined as above for Formula I. As shown above in Structure I-A, the compound has a total charge of negative three. Therefore, three cations (i.e., 3 M+) are present to balance the charge of the compound (i.e., the total positive charge equals the total negative charge).

An example of Structure I-A includes the following compound:

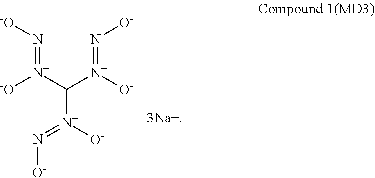

The compound can have a molecular weight below 500 g/mol, not including the associated cation (e.g., the associated pharmaceutically-acceptable cation). For example, the compound can have a molecular weight of 450 g/mol or less, 400 g/mol or less, 350 g/mol or less, 300 g/mol or less, 250 g/mol or less, or 200 g/mol or less. Optionally, the molecular weight of the compound, excluding the associated cation, can be from 100 g/mol to below 500 g/mol, from 120 g/mol to 450 g/mol, from 150 g/mol to 400 g/mol, or from 175 g/mol to 350 g/mol.

Additional details regarding the mechanism of action of the compounds described herein, including their nitric oxide delivery properties, and advantageous properties of the compounds (e.g., storage stability) are described in PCT/US2021/016841, entitled “Nitric Oxide-Releasing Antibacterial Compounds, Formulations, and Methods Pertaining Thereto;” PCT/US2021/016854, entitled “Nitric Oxide-Releasing Antibacterial Compounds, Formulations, and Methods Pertaining Thereto;” and/or PCT/US2021/016869, entitled “Nitric Oxide-Releasing Antibacterial Compounds, Formulations, and Methods Pertaining Thereto;” each of which are incorporated herein by reference in their entireties.

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bonded through a single, terminal ether linkage. The term aryloxy as used herein is an aryl group bonded through a single, terminal ether linkage. Likewise, the terms alkenyloxy, alkynyloxy, heteroalkyloxy, heteroalkenyloxy, heteroalkynyloxy, heteroaryloxy, cycloalkyloxy, and heterocycloalkyloxy as used herein are an alkenyloxy, alkynyloxy, heteroalkyloxy, heteroalkenyloxy, heteroalkynyloxy, heteroaryloxy, cycloalkyloxy, and heterocycloalkyloxy group, respectively, bonded through a single, terminal ether linkage.

The term hydroxy as used herein is represented by the formula —OH.

The terms amine or amino as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, aryloxy, amino, alkyl, alkenyl, alkynyl, aryl, heteroalkyl, heteroalkenyl, heteroalkynyl, heteroaryl, cycloalkyl, or heterocycloalkyl molecules used herein can be substituted or unsubstituted. As used herein, the term substituted includes the addition of an alkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens, i.e., commensurate with its saturation level, with no substitutions, e.g., linear decane (—(CH₂)₉—CH₃).

The nitric oxide releasing compounds described herein are pH-triggered NO-releasing donors (also referred to herein as NO-releasing compounds or NO-releasing agents). Reacting with protons under physiological conditions (e.g., 37° C., pH 7.4), 1 mole of Compound 1 (MD3) generates two moles of NO and 2 to 3 moles of nitroxyl compounds.

In several embodiments, the NO-releasing compounds are stable at a variety of temperatures from frozen to room temperature 25° C. (e.g., −20° C. 0° C., 5° C., 20° C., etc.) and are stable for prolonged storage periods (e.g., 10 hours, 20 hours, 22 hours, 25 hours, 30 hours, etc., days such as 1 day, 3 days, 5 days, 6 days, 7 days, 15 days, 30 days, 45 days, etc., weeks such as 1 week, 2 weeks, 3 weeks, 4 weeks, 6 weeks, 8 weeks, etc., months such as 1 month, 2 months, 3 months, 4 months, 5 months, 6 months, etc., or even years (1 year, 2 years, or greater)).

In some cases, the compound has a total releasable NO storage in a range of 0.1 μmol-23.0 μmol of NO per mg of the compound (e.g., from 0.1 μmol to 15 μmol per mg of the compound, from 0.5 μmol to 7.5 μmol per mg of the compound, from 1 μmol to 7.0 μmol per mg of the compound, from 1.5 μmol to 6.5 μmol per mg of the compound, from 2.0 μmol to 6.0 μmol per mg of the compound, from 2.5 μmol to 5.5 μmol per mg of the compound, or from 3.0 μmol to 5.0 μmol per mg of the compound). For example, the total releasable NO storage of the compounds for use in the composition can be 0.1 μmol, 0.2 μmol, 0.3 μmol, 0.4 μmol, 0.5 μmol, 0.6 μmol, 0.7 μmol, 0.8 μmol, 0.9 μmol, 1.0 μmol, 1.1 μmol, 1.2 μmol, 1.3 μmol, 1.4 μmol, 1.5 μmol, 1.6 μmol, 1.7 μmol, 1.8 μmol, 1.9 μmol, 2.0 μmol, 2.1 μmol, 2.2 μmol, 2.3 μmol, 2.4 μmol, 2.5 μmol, 2.6 μmol, 2.7 μmol, 2.8 μmol, 2.9 μmol, 3.0 μmol, 3.1 μmol, 3.2 μmol, 3.3 μmol, 3.4 μmol, 3.5 μmol, 3.6 μmol, 3.7 μmol, 3.8 μmol, 3.9 μmol, 4.0 μmol, 4.1 μmol, 4.2 μmol, 4.3 μmol, 4.4 μmol, 4.5 μmol, 4.6 μmol, 4.7 μmol, 4.8 μmol, 4.9 μmol, 5.0 μmol, 5.1 μmol, 5.2 μmol, 5.3 μmol, 5.4 μmol, 5.5 μmol, 5.6 μmol, 5.7 μmol, 5.8 μmol, 5.9 μmol, 6.0 μmol, 6.1 mol, 6.2 μmol, 6.3 μmol, 6.4 μmol, 6.5 μmol, 6.6 μmol, 6.7 μmol, 6.8 μmol, 6.9 μmol, 7.0 μmol, 7.1 μmol, 7.2 μmol, 7.3 μmol, 7.4 μmol, 7.5 μmol, 7.6 μmol, 7.7 μmol, 7.8 μmol, 7.9 μmol, 8.0 μmol, 8.1 μmol, 8.2 μmol, 8.3 μmol, 8.4 μmol, 8.5 μmol, 8.6 μmol, 8.7 μmol, 8.8 μmol, 8.9 μmol, 9.0 μmol, 9.1 μmol, 9.2 μmol, 9.3 μmol, 9.4 μmol, 9.5 μmol, 9.6 μmol, 9.7 μmol, 9.8 μmol, 9.9 μmol, 10.0 μmol, 10.1 μmol, 10.2 μmol, 10.3 μmol, 10.4 μmol, 10.5 μmol, 10.6 μmol, 10.7 μmol, 10.8 μmol, 10.9 μmol, 11.0 μmol, 11.1 μmol, 11.2 μmol, 11.3 μmol, 11.4 μmol, 11.5 μmol, 11.6 μmol, 11.7 μmol, 11.8 μmol, 11.9 μmol, 12.0 μmol, 12.1 μmol, 12.2 μmol, 12.3 μmol, 12.4 μmol, 12.5 μmol, 12.6 μmol, 12.7 μmol, 12.8 μmol, 12.9 μmol, 13.0 μmol, 13.1 μmol, 13.2 μmol, 13.3 μmol, 13.4 μmol, 13.5 μmol, 13.6 μmol, 13.7 μmol, 13.8 μmol, 13.9 μmol, 14.0 μmol, 14.1 μmol, 14.2 μmol, 14.3 μmol, 14.4 μmol, 14.5 μmol, 14.6 μmol, 14.7 μmol, 14.8 μmol, 14.9 μmol, 15.0 μmol, 15.1 μmol, 15.2 μmol, 15.3 μmol, 15.4 μmol, 15.5 μmol, 15.6 μmol, 15.7 μmol, 15.8 μmol, 15.9 μmol, 16.0 μmol, 16.1 μmol, 16.2 μmol, 16.3 μmol, 16.4 μmol, 16.5 μmol, 16.6 μmol, 16.7 μmol, 16.8 μmol, 16.9 μmol, 17.0 μmol, 17.1 μmol, 17.2 μmol, 17.3 μmol, 17.4 μmol, 17.5 μmol, 17.6 μmol, 17.7 μmol, 17.8 μmol, 17.9 μmol, 18.0 μmol, 18.1 μmol, 18.2 μmol, 18.3 μmol, 18.4 μmol, 18.5 μmol, 18.6 μmol, 18.7 μmol, 18.8 μmol, 18.9 μmol, 19.0 μmol, 19.1 μmol, 19.2 μmol, 19.3 μmol, 19.4 μmol, 19.5 μmol, 19.6 μmol, 19.7 μmol, 19.8 μmol, 19.9 μmol, 20.0 μmol, 20.1 μmol, 20.2 μmol, 20.3 μmol, 20.4 μmol, 20.5 μmol, 20.6 μmol, 20.7 μmol, 20.8 μmol, 20.9 μmol, 21.0 μmol, 21.1 μmol, 21.2 μmol, 21.3 μmol, 21.4 μmol, 21.5 μmol, 21.6 μmol, 21.7 μmol, 21.8 μmol, 21.9 μmol, 22.0 μmol, 22.1 μmol, 22.2 μmol, 22.3 μmol, 22.4 μmol, 22.5 μmol, 22.6 μmol, 22.7 μmol, 22.8 μmol, 22.9 μmol, or 23.0 μmol per mg of the compound.

The compound can have a total duration of NO release, upon activation, in a range of 0.1-60 hours. In some cases, the NO release may occur over a period of about 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, or 60 hours. In some embodiments, within 2 hours of being added to a PBS buffer solution, the compounds release greater than or equal to about: 25%, 50%, 75%, 85%, 90%, 95%, 100%, or ranges including and/or spanning the aforementioned values, their total wt. % of bound NO. Optionally, the compound has a total NO release of 0.1-8.0 μmol of NO per mg of the compound after 4 hours of the initiation of NO release (also referred to as “activation”).

In some embodiments, the compounds have a release rate per hour using chemiluminescent based nitric oxide detection of less than or equal to about: 0.2%, 0.5%, 1.0%, 1.5%, 2.5%, 5.0%, 10%, or ranges including and/or spanning the aforementioned values.

Optionally, the compounds described herein has a NO release half-life in the range of 0.01-24 hours. In several embodiments, the NO release half-life is equal to or at least about: 0.01 hours, 0.1 hours, 0.25 hours, 0.5 hours, 1 hour, 2 hours, 3 hours, 4 hours, 5 hours or ranges including and/or spanning the aforementioned values. In some embodiments, the NO release occurs in less than or equal to about: 0.01 hours, 0.1 hours, 0.25 hours, 0.5 hours, 1 hour, 2 hours, 3 hours, 4 hours, 5 hours, 10 hours, 15 hours, 20 hours, 24 hours or ranges including and/or spanning the aforementioned values.

The high purity nitric oxide releasing compound product has a purity of at least 97 area % as measured by high pressure liquid chromatography. In other words, the nitric oxide releasing compound represents 97% of the measurable content (e.g., mass) of the product. Optionally, the purity of the high purity nitric oxide releasing compound product is 97 area % or greater, 97.5 area % or greater, 98 area % or greater, 98.5 area % or greater, 99 area % or greater, or 99.5 area % or greater.

The area %, or peak area, as measured by high pressure liquid chromatography or other analytical techniques, can be converted to concentration using known methods in analytical chemistry. For example, a calibration curve can be prepared from peak areas of known concentrations of the high purity nitric oxide releasing compound product, and the calibration curve can be used to determine the concentration of the sample based on the sample's peak area. In some cases, the nitric oxide releasing compound represents 97% w/w, 97.5% w/w, 98% w/w, 98.5% w/w, 99% w/w, 99.5% w/w, or greater than 99.5% w/w of the product.

The remaining components of the high purity nitric oxide releasing compound product can optionally be residual solvents used during the manufacturing/purification process or impurities of the nitric oxide releasing compound.

In some examples, the high purity nitric oxide releasing compound product has a residual solvent level of less than 1.5% w/w. Exemplary solvents that can be present as the residual solvent can include an alcohol, such as methanol (MeOH), ethanol (EtOH), n-propanol, isopropyl alcohol (also referred to as “isopropanol”; IPA), and/or isobutanol. Optionally, the solvent that can be present in the product as residual solvent can include a ketone, such as acetone, dihydroxyacetone, and/or dimethyl malonate. As understood to those of ordinary skill in the art, the residual solvent(s) will vary based on the solvent(s) used in the manufacturing method. Optionally, the residual solvent level remaining in the high purity nitric oxide releasing compound product is less than 1.5% w/w, less than 1.4% w/w, less than 1.3% w/w, less than 1.2% w/w/, less than 1.1% w/w, less than 1.0% w/w, less than 0.9% w/w, less than 0.8% w/w, less than 0.7% w/w, less than 0.6% w/w, less than 0.5% w/w, less than 0.4% w/w, less than 0.3% w/w, less than 0.2% w/w, or less than 0.1% w/w.

In some examples, the high purity nitric oxide releasing compound product includes one or more impurities. In general, the impurity level, in total, of the one or more impurities is less than 1 area % as measured by high pressure liquid chromatography. For example, the one or more impurities can be less than 0.9 area %, less than 0.8 area %, less than 0.7 area %, less than 0.6 area %, less than 0.5 area %, less than 0.4 area %, less than 0.3 area %, less than 0.2 area %, or less than 0.1 area %.

In some cases, the impurity is a methane bis-diazeniumdiolate, which is represented by Structure I-B, as shown below:

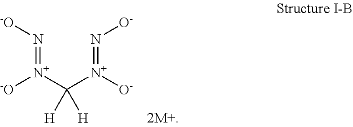

In Structure I-B. M+ is defined as above for Formula I. As shown above in Structure I-B, the compound has a total charge of negative two. Therefore, two cations (i.e., 2 M+) are present to balance the charge of the compound (i.e., the total positive charge equals the total negative charge).

An example of Structure I-B includes the following compound, which is also referred to herein as MD2:

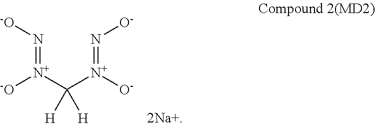

Optionally, the high purity nitric oxide releasing compound product is crystalline in form. The product can optionally be prepared as a “product preparation,” which can optionally include packing and sealing the compound in a container. The product preparation can also include an aqueous solvent, such as water. The preparation can be stored for a period of time (e.g., one week or more, two weeks or more, three weeks or more, four weeks or more, one month or more, two months or more, three months or more, six months or more, one year or more, two years or more, and the like) until selected for use. Optionally, the high purity nitric oxide releasing compound products can be stored at a temperature of −20° C. or greater (e.g., −10° C. or greater, 0° C. or greater, 5° C. or greater, 10° C. or greater, 15° C. or greater, 20° C. or greater, or 25° C. or greater).

Optionally, the high purity nitric oxide releasing compound product preparations as described herein exhibit a heat of decomposition of less than 300 J/g (e.g., less than 275 J/g, less than 250 J/g, less then 225 J/g, less than 200 J/g, less than 175 J/g, less than 150 J/g, less than 125 J/g, less than 100 J/g, less than 75 J/g, or less than 50 J/g).

### Manufacturing Method

A method of manufacturing high purity nitric oxide releasing compound products is provided herein, including the products described above. Optionally, the products can be prepared as a preparation in an aqueous solvent, such as water. The method includes preparing in a vessel a reaction mixture comprising a hydroxide or a hydroxide donor and one or more reaction mixture solvents.

In some cases, the reaction mixture comprises a hydroxide selected from the group consisting of sodium hydroxide, potassium hydroxide, lithium hydroxide, calcium hydroxide, magnesium hydroxide, or ammonium hydroxide. In other cases, the reaction mixture comprises a hydroxide donor selected from the group consisting of sodium methoxide, lithium methoxide, potassium methoxide, calcium methoxide, magnesium methoxide, or ammonium methoxide. The one or more reaction mixture solvents can include an alcohol. Suitable alcohols for use as the reaction mixture solvent can include methanol, ethanol, n-propanol, isopropanol, and/or isobutanol. Optionally, the reaction mixture solvent includes a ketone, such as acetone, dihydroxyacetone, and/or dimethyl malonate.

After preparing the reaction mixture, nitric oxide is introduced into the vessel under an inert atmosphere (e.g., nitrogen) to obtain a suspension comprising a nitric oxide releasing compound. The nitric oxide pressure can be adjusted, as determined by one of ordinary skill in the art. Suitable nitric oxide pressures can include 5 bar or higher, 10 bar or higher, 15 bar or higher, 20 bar or higher, or 25 bar or higher. The reaction mixture is stirred during the introduction of the nitric oxide gas. Once the nitric oxide releasing compound forms, the reaction mixture becomes a suspension. The suspension is filtered to obtain the nitric oxide releasing compound as a solid.

Following the filtration step, the nitric oxide releasing compound solid is then dissolved in an aqueous solution (e.g., water) to form a dissolved nitric oxide releasing compound solution. Following the dissolution step, the nitric oxide releasing compound solid is recrystallized from the dissolved nitric oxide releasing compound solution to form a recrystallized nitric oxide releasing compound product. The recrystallizing includes a step of adding methanol to the dissolved nitric oxide releasing compound solution. In some instances, an alternative alcohol can be used in place of methanol. The methanol or other recrystallizing alcohol can be added by dropwise addition.

Optionally, a nitric oxide releasing compound seed can be added during the step of adding methanol (or other solvent) to the dissolved nitric oxide releasing compound solution. Seed crystals help control the nucleation and growth stages of the recrystallization process. In some cases, the timing of introducing the seed is controlled to result in desired characteristics (e.g., the desired product form) of the products. Optionally, adding the nitric oxide releasing compound seed is initiated after a concentration of methanol in the dissolved nitric oxide releasing compound solution reaches at least 15% (v/v) or at least 20% (v/v). The seed can be added to the dissolved nitric oxide releasing compound solution as a powder, or optionally as a suspension in a solvent or a mixture of solvents. Suitable solvents include, for example, water and methanol. The ratio of methanol to water in the solvent mixture for the suspension can be controlled to prevent the nitric oxide releasing compound from becoming amorphous. Optionally, the ratio of methanol to water in the solvent mixture for the suspension can be from 10:1 to 1:1 methanol to water. For example, the ratio of methanol to water can be 7:3 or 4:1.

Once recrystallized, the recrystallized nitric oxide releasing compound product is then filtered, and the filtered recrystallized nitric oxide releasing compound product is washed with a mixture of washing solvents to form a washed nitric oxide releasing compound product. The mixture of washing solvents can include one or more solvents, such as one or more alcohols. In some cases, the washing solvent is methanol. In some cases, the washing solvents include methanol and one or more additional alcohols. The washing solvents are selected such that the solvents are miscible. Optionally, the mixture of washing solvents comprises methanol and a C₂-C₂₀ alcohol, as long as the selected C₂-C₂₀ alcohol is miscible with methanol. The C₂-C₂₀ alcohol can be, for example, ethanol, propanol, isopropanol, butanol, or isobutanol. The mixture of washing solvents can be present in a ratio of methanol to C₂-C₂₀ alcohol of 1:1 to 1:10 (e.g., 1:1, 1:2, 1:3, 1:4, 1:5, 1:6, 1:7, 1:8, 1:9, or 1:10 methanol to miscible C₂-C₂₀ alcohol).

The washed nitric oxide releasing compound product is then dried to form a dried nitric oxide releasing compound product. Optionally, the drying is performed under vacuum. The resulting dried product is high in purity, having minimal process impurities and residual solvents. In some examples, the high purity nitric oxide releasing compound product has a purity of at least 97 area % as measured by high pressure liquid chromatography, a residual solvent level of less than 1.5% w/w and an impurity level of less than 1 area % as measured by high pressure liquid chromatography. In some cases, the dried nitric oxide releasing compound product is a crystalline form. The crystalline product can be present as a loose or compact powder. Optionally, the dried nitric oxide releasing compound product can be present in an amorphous form.

To form the product preparation, which as described above is suitable for storage, the dried nitric oxide releasing compound product is dissolved in an aqueous solvent (e.g., water) to form the high purity nitric oxide releasing compound product preparation.

As used herein the terms treatment, treat, or treating refer to a method of reducing one or more symptoms of a disease or condition. Thus, in the disclosed method, treatment can refer to a 10%, 20%, 30%, 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs of the disease in a subject as compared to a control. As used herein, control refers to the untreated condition. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 10%, 20%, 30%, 40%, 50%, 60%, 70%, 80%, 90% or greater as compared to a control level. Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs, and goats. Non-mammals include, for example, fish and birds.

Throughout this application, various publications are referenced. The disclosures of these publications in their entireties are hereby incorporated by reference into this application.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the subject matter described herein which are apparent to one skilled in the art.

### Example 1: Nitric Oxide Releasing Compound Product Preparation

MD3, a nitric oxide releasing compound as described herein, was prepared in methanol according to the reaction shown in Scheme 1 and detailed below.

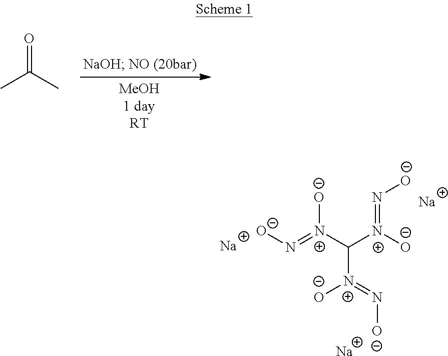

Sodium hydroxide (358.6 g) was added to 9.3 L of methanol and stirred until dissolved. Acetone (166 mL) was added and the solution was allowed to stir for five minutes. The resulting solution was charged into a 10 L autoclave reactor under vacuum and the reactor was purged with nitrogen and vented three times. The reaction solution was stirred at a temperature of 22° C. and nitric oxide was added to the reactor at a pressure of 20 bar for a period of at least 24 hours. The reaction progress was monitored by observing the NO consumption via an Additel gauge. Once the reaction was deemed complete, the system was purged with nitrogen three times.

The resulting reaction mixture suspension was filtered under nitrogen, and the obtained MD3 solid was redissolved in water. The MD3 was then recrystallized from the solution by slowly adding methanol to the MD3 solution. Approximately 6 g of MD3 crystals were suspended per 100 mL of methanol. Once the methanol concentration reached 20% v/v, the MD3 suspension was added to the filter/dryer to seed the recrystallization process. After adding the MD3 seed material, methanol was added until the final ratio of methanol to water was 4:1 (80% v/v). The recrystallized MD3 was filtered, and a mixture of isopropyl alcohol and methanol (75/25% v/v) was added to the filter dryer to remove excess water and other process impurities. The MD3 was filtered and then dried under vacuum. Once the drying was complete, 6 L of water for injection (WFI) water was added to the filter dryer vessel to dissolve the powder. Once all powder dissolved, the solution was dispensed into clean containers suitable for storage. The solution was stored at −20° C.

### Example 2: Reverse Addition Reprecipitation Studies

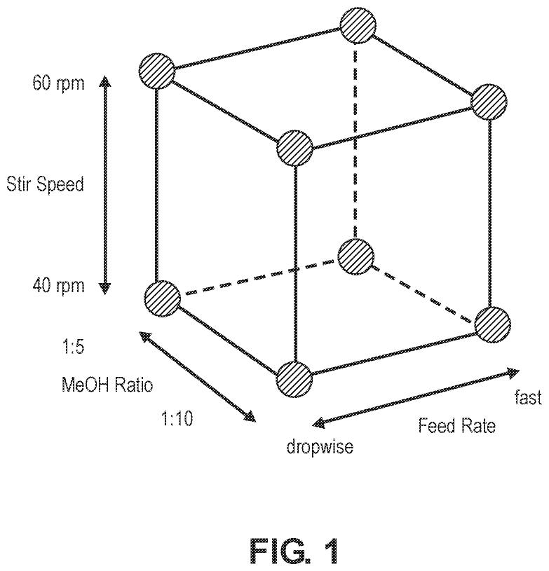

An experimental design (see FIG. 1) was generated to investigate a reverse addition reprecipitation process. The reverse addition process generally involved reprecipitating a dissolved solute by adding the solute/solvent (e.g., water) solution to an anti-solvent (e.g., methanol). Reverse addition processes typically generate small amorphous particles. The reverse addition process experiments evaluated the impact of the final solvent to anti-solvent ratio (e.g., 1:5 vs 1:10), the feed rate of aqueous solution to anti-solvent (e.g., drop-wise vs. fast), and the stir speed on the purity and residual solvent content of the reprecipitated material. Of note, the fast feed rate is equivalent to pouring the MD3 solution into the anti-solvent while the dropwise addition was performed as implied.

Crude MD3 material, prepared by reacting acetone with NO under basic conditions (as described above in Example 1), was isolated, taken up in DI water at a concentration of 150 mg/mL MD3, and stored in containers. The aqueous MD3 solution was portioned into: 1 L aliquots and reprecipitated per the conditions shown in FIG. 1. Following the reprecipitation step, the solid MD3 was washed in methanol. A small sample of each of the reprecipitated MD3 samples was set aside prior to the wash step to observe changes in the impurity MD2 and residual solvent content in each subsequent step of the process. The purified MD3 product was isolated via vacuum filtration and samples were dried overnight in vacuo prior to analysis.

### Results and Discussion

The product purity profiles of MD3 obtained using the different reverse addition reprecipitation conditions were assessed. MD3 and MD2 percentages were determined via high pressure liquid chromatography (HPLC) and residual solvent content via head-space gas chromatography. The powder samples were also examined under a microscope to qualitatively determine whether the material was crystalline or amorphous. The results are presented in Table 1.

TABLE 1
MD3/MD2 and residual solvent content from the
reverse addition reprecipitation samples

| | | Crystalline | MD3 | MD2 | % |
|---|---|---|---|---|---|
| Reprecipitation | MeOH | or | Area | Area | MeOH |
| Conditions | Wash | Amorphous | % | % | (wt/wt) |
| 1:5, 40 rpm, | No | Amorphous | 97.3 | 1.5 | 0.1 |
| dropwise addition | Yes | Amorphous | 97.8 | 0.8 | 3.0 |
| 1:5, 40 rpm, | No | Amorphous | 96.8 | 1.8 | 1.4 |
| fast addition | Yes | Amorphous | 98.2 | 0.6 | 2.8 |
| 1:5, 60 rpm, | No | Amorphous | 96.3 | 2.0 | 0.1 |
| dropwise addition | Yes | Amorphous | 97.0 | 0.8 | 3.1 |
| 1:5, 60 rpm, | No | Amorphous | 97.3 | 1.5 | 0.4 |
| fast addition | Yes | Amorphous | 98.2 | 0.7 | 2.8 |

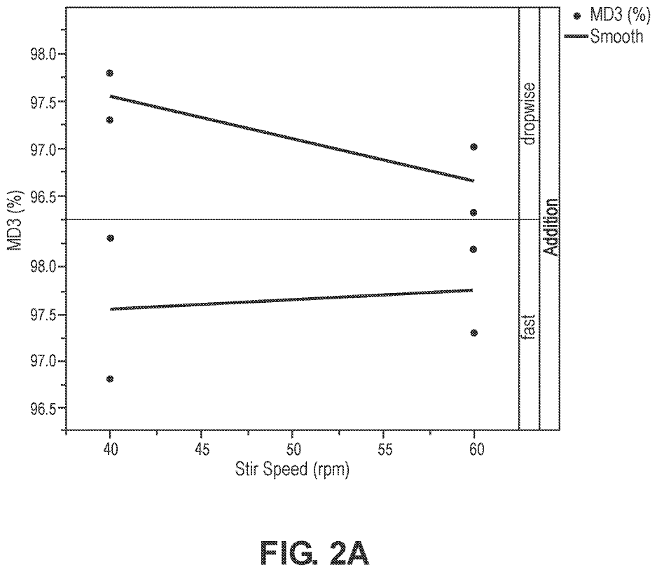

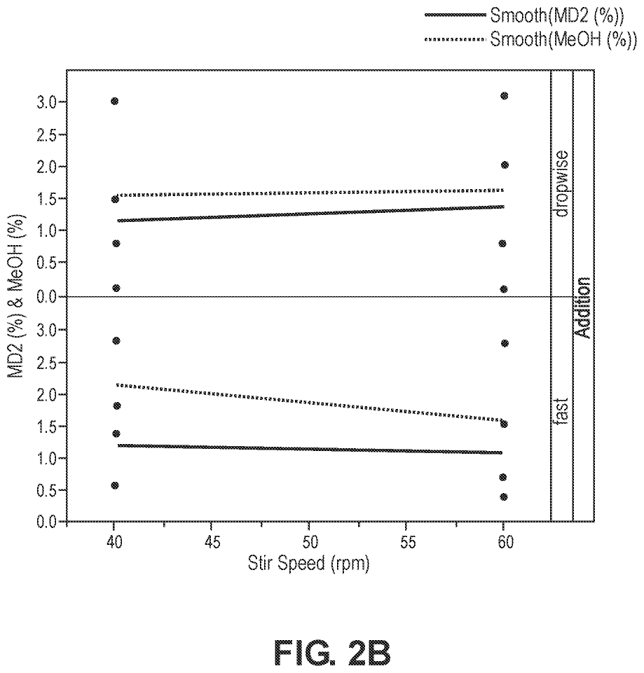

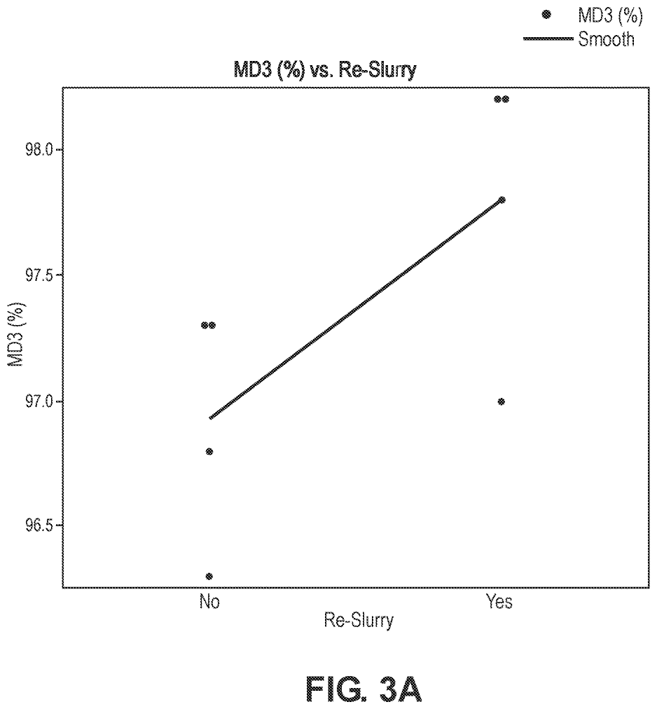

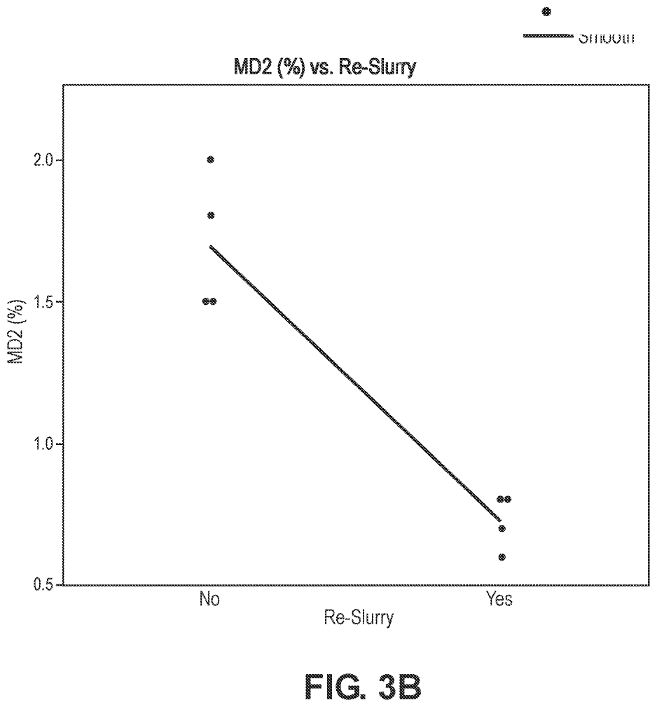

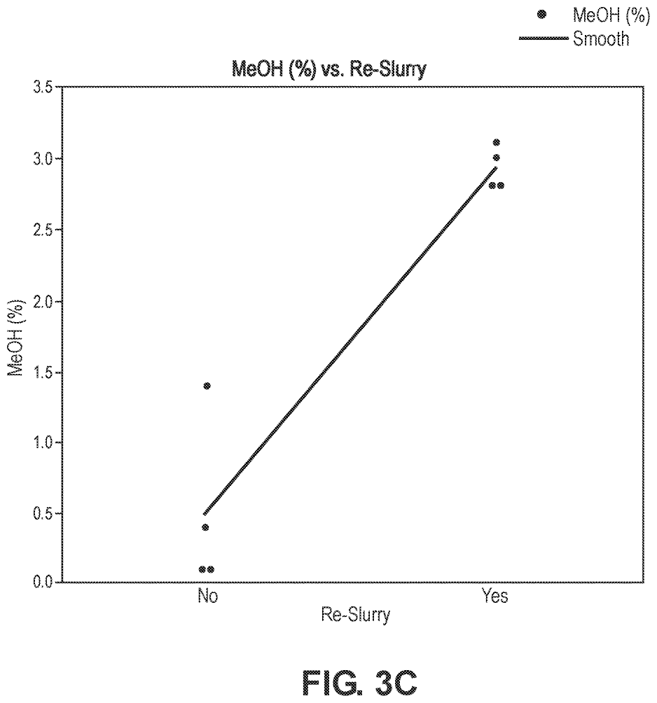

The 1:10 MD3 solution to MeOH ratio resulted in fine particles that passed through the filter paper during the isolation process. As a consequence, product was lost and no samples were obtained for analysis for those conditions. Based on this outcome, the use of 1:10 ratio was ruled out as a viable process parameter. Samples reprecipitated in a 1:5 MD3 solution to MeOH ratio were successfully isolated and analyzed. These samples were examined under a microscope and the resulting particles from the reprecipitation and wash steps were found to be very fine and amorphous in nature. Neither feed rate nor stir speed had a significant impact on minimizing MD2 content or residual solvent content (FIGS. 2A-2B); however, the addition of the wash step had an observable effect on product purity (FIGS. 3A-3C). The wash step decreased the MD2 content of the reprecipitated MD3 from almost 2% down to 0.7%. While the wash step was successful in minimizing the MD2 impurity, it increased the residual solvent content from 0.1 to 3.1%.

### Example 3: Normal Addition Reprecipitation Studies and Seeding Impact

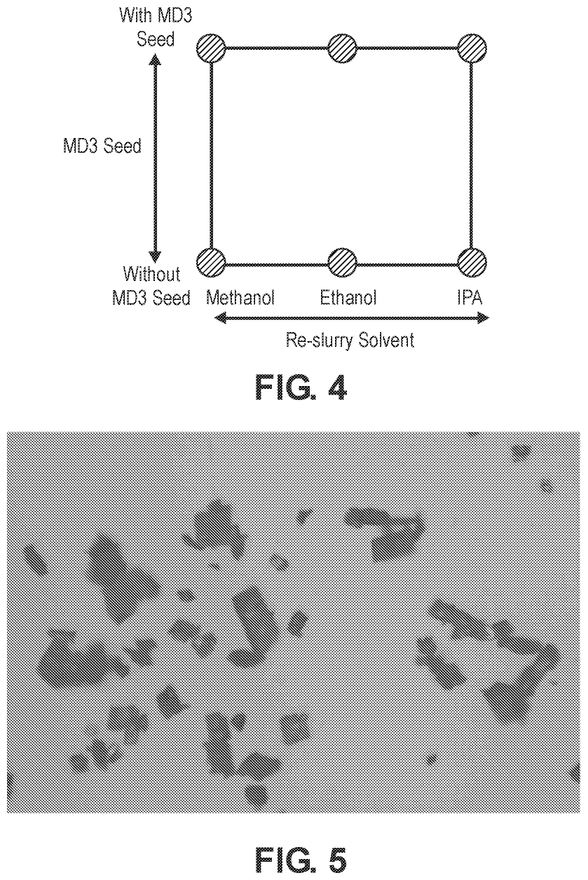

An experimental design (see FIG. 4) was generated to investigate a normal addition process and the impact of seeding vs. not seeding to induce precipitation. The normal addition reprecipitation process generally involved reprecipitating a dissolved solute by adding an anti-solvent to a solute/solvent (e.g., water) solution at a controlled rate. Normal addition processes are typically used to generate larger, crystalline particles. In addition to seeding, several wash solvents (e.g., methanol, ethanol, or isopropyl alcohol) were also evaluated for their ability to be dried from the final precipitated material. A 1:5 MD3 solution to methanol ratio, a dropwise addition rate, and a 200 rpm stir speed were used for each run. Once reprecipitated, the product was isolated via vacuum filtration and samples were dried overnight in vacuo prior to analysis.

### Results and Discussion

Reprecipitation using a normal addition method was evaluated both with and without the use of MD3 seed crystals. Seed crystals were used to help control the nucleation and growth stages of the recrystallization process. The timing for adding the seed crystal was determined by observing when the MD3 water solution began to turn turbid. After the seed crystals were added, additional MeOH was added until the target water/MeOH ratios were reached. The results are summarized in Table 2.

TABLE 2
MD3/MD2 and residual solvent content from
the normal addition reprecipitation samples

| | | Crystalline | MD3 | MD2 | | | |
|---|---|---|---|---|---|---|---|
| Reprecipitation | Wash | or | Area | Area | % MeOH | % EtOH | % IPA |
| Conditions | Solvent | Amorphous | % | % | (wt/wt) | (wt/wt) | (wt/wt) |
| 1:5, 200 rpm, | None | Crystalline | 97.6 | 1.0 | 0.1 | — | — |
| dropwise | | | | | | | |
| addition, | | | | | | | |
| without seeded | | | | | | | |
| MD3 | | | | | | | |
| 1:5, 200 rpm, | None | Crystalline | 97.9 | 0.8 | 0.1 | — | — |
| dropwise | MeOH | Amorphous | 98.2 | 0.3 | 2.8 | — | — |
| addition, with | EtOH | Crystalline | 97.6 | 0.9 | 0.1 | 0.2 | — |
| seeded MD3 | IPA | Crystalline | 98.2 | 0.8 | 0.0 | — | 0.1 |

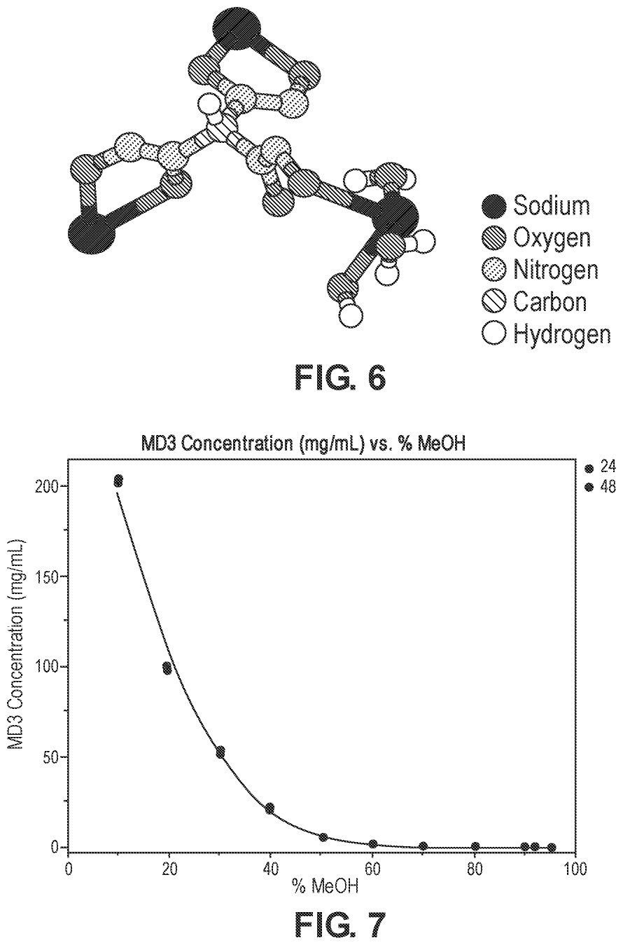

The normal addition method of reprecipitation yielded MD3 with lower MD2 levels (<1.0%) than the material isolated from a reverse addition reprecipitation (>1.5%) and similar residual solvent content (<0.5%). The normal addition method also formed large crystals as opposed to fine amorphous solids (FIG. 5). A crystal structure analysis was also performed. The results of the crystal structure analysis showed that MD3 existed as a trihydrate, with the three H₂O molecules bound to only one of the sodium atoms (FIG. 6).

MD3 obtained from normal addition without seed crystals could not be washed as the product formed on the walls of the glass beaker used for the reprecipitation and could not be recovered.

Washing the recrystallized MD3 with MeOH resulted in an increase in residual solvent content (from 0.1 to 2.8%). This was an unexpected result, but it appears that the MD3 product converts back to an amorphous material in the presence of pure MeOH. Washing the recrystallized MD3 in either ethanol (EtOH) or isopropyl alcohol (IPA) maintained the low residual solvent content of the non-washed MD3 and the crystallinity of the MD3 was retained when washing in either EtOH or IPA. However, neither EtOH nor IPA effectively reduced the MD2 impurity content any further than the levels obtained during the recrystallization step. Because MD2 is more soluble in MeOH than IPA or EtOH, various ratios of IPA and MeOH were evaluated with the aim of further reducing MD2 in the final product (Table 3) but also maintaining the crystal structure of the recrystallized product. IPA was selected as the wash solvent to generate the results.

TABLE 3
MD3/MD2 and residual solvent content for MD3 washed in IPA-MeOH.

| | IPA:Methanol | Crystalline | MD3 | MD2 | | | |
|---|---|---|---|---|---|---|---|
| Reprecipitation | as Wash | or | Area | Area | % MeOH | % EtOH | % IPA |
| Conditions | Solvent | Amorphous | % | % | (wt/wt) | (wt/wt) | (wt/wt) |
| 1:5, 200 rpm, | None | Crystalline | 97.6 | 1.1 | 0.10 | — | — |
| dropwise | 1:1 | Amorphous | 97.1 | 1.5 | 0.60 | — | ND |
| addition, with | 7:3 | Crystalline | 97.3 | 1.4 | 0.02 | — | ND |
| seeded MD3 | 9:1 | Crystalline | 97.3 | 1.4 | 0.01 | — | 0.02 |

There was no significant reduction in either MD2 levels or residual solvent content when different ratios of IPA and MeOH was used for the wash step. MD3 retained its crystalline structure when higher IPA content was used (i.e., 70% and 90% IPA). When IPA and MeOH were combined in equal parts the crystallinity of MD3 was lost.

### Example 4: Manufacturing Scale-Up

A scale-up experiment was conducted to verify that the conditions identified for recrystallizing MD3 could be transferred to a 10-L manufacturing scale. To prepare for running this batch, the MD3 solubility curve was measured for the MeOH:water solvent system. The solubility curve is shown in FIG. 7.

A first large scape reprecipitation was performed using EtOH. The large scale reprecipitation and wash process yielded similar results (Table 4) to the smaller scale experiment with ethanol (Table 2). The final product isolated in powder form was crystalline with minimal MD2 and EtOH content (0.5% and 0.3%, respectively), with no MeOH detected.

TABLE 4
MD3/MD2 and residual solvent content for bulk (1.5 L) MD3 collection

| | Crystalline | | MD3 | MD2 | | |
|---|---|---|---|---|---|---|
| Reprecipitation | or | | Area | Area | % MeOH | % EtOH |
| Conditions | Amorphous | Form | % | % | (wt/wt) | (wt/wt) |
| 1:5, 50 rpm, dropwise | Crystalline | Powder | 98.4 | 0.5 | ND | 0.31 |
| addition, with seeded | | Solution | 98.6 | | | |
| MD3 | | | | | | |

To ensure that similar results would be observed with a final IPA/MeOH wash step, crude MD3 was re-processed using the recrystallization process.

The large scale reprecipitation and wash results of MD3 (Table 5) agreed with the small-scale reprecipitation experiment that used a final 7:3 IPA and MeOH wash step. The final product isolated in powder form was crystalline with minimal MD2, IPA, and MeOH content (0.3%, 0.08%, and 0.3% respectively).

TABLE 5
MD3/MD2 and residual solvent content for bulk (6 L) MD3 collection

| | Crystalline | | MD3 | MD2 | | |
|---|---|---|---|---|---|---|
| Reprecipitation | or | | Area | Area | % MeOH | % IPA |
| Conditions | Amorphous | Form | % | % | (wt/wt) | (wt/wt) |
| 1:5, 50 rpm, dropwise | Crystalline | Powder | 98.8 | 0.3 | 0.08 | 0.30 |
| addition, with seeded | | Solution | 99.0 | | | |
| MD3 | | | | | | |

A third full-scale 10-L batch was prepared. Since suspension in MeOH converts the crystalline MD3 into amorphous material over time, the seed crystal in this experiment was resuspended in an 8:2 MeOH:H₂O mixture. The MeOH addition rate was also ramped up from 200 mL/min to 500 mL/min for the final MeOH addition step (from 7 L to 21 L mark) since most of the MD3 will have precipitated out of solution by then.

The results (Table 6) agree with the first two large scale reprecipitation and wash experiments. The results also agree with the small-scale reprecipitation experiment with a final 7:3 IPA and MeOH wash step. The final product isolated in powder form were crystalline with minimal MD2, IPA, and MeOH content (0.3%, 0.08%, and 0.3% respectively).

TABLE 6
MD3/MD2 and residual solvent content for VT-21-040.

| | Crystalline | | MD3 | MD2 | | |
|---|---|---|---|---|---|---|
| Reprecipitation | or | | Area | Area | % MeOH | % IPA |
| Conditions | Amorphous | Form | % | % | (wt/wt) | (wt/wt) |
| 1:5, 50 rpm, dropwise | Crystalline | Powder | 99.2 | 0.4 | 0.02 | 0.06 |
| addition, with seeded | | Solution | 99.3 | | | |
| MD3 | | | | | | |

The final process defined in the smaller scale reprecipitation experiments translated well in the bulk reprecipitation of MD3. The optimized reprecipitation and wash conditions yielded MD3 crystals with high MD3 purity for both powder (98%) and solution (99%) forms, with minimal impurities (i.e., MD2, MeOH, and IPA).

Of the parameters studied, the method for reprecipitation (normal vs reverse) had the greatest impact on the purity of the MD3 product obtained in the collection process. The normal addition method of reprecipitation produced MD3 crystals with low MD2 and residual solvent content. Further washing in an IPA and MeOH solvent mixture ensured low levels of impurities for the final MD3 product. The initial bulk collection also yielded the purest MD3 batch manufactured thus far, with product purity up to ~98% MD3, with residual solvent levels well below recommended International Council on Harmonisation (ICH) guidelines.

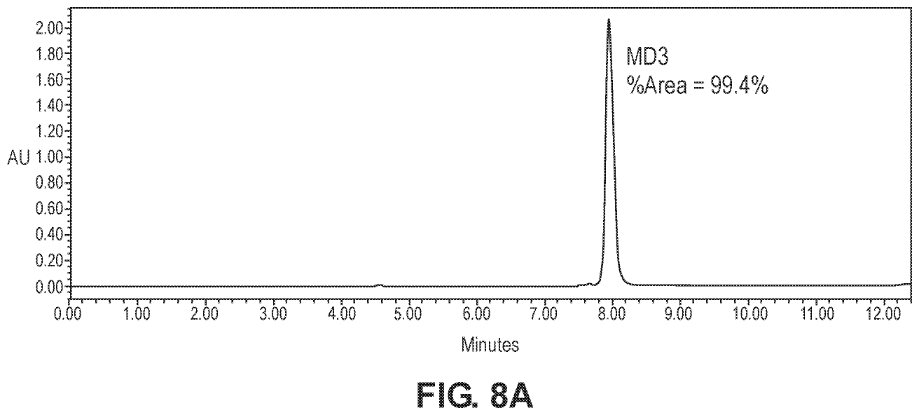

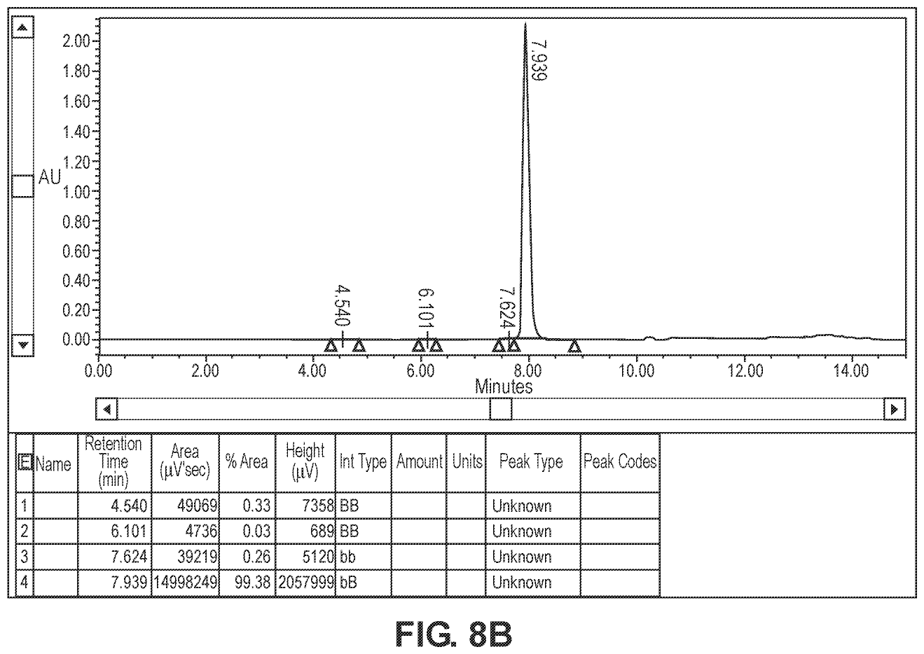

High pressure liquid chromatography chromatograms, obtained from recrystallized MD3 prepared according to the methods described in Example 4, are shown in FIGS. 8A and 8B. As shown in FIGS. 8A and 8B, the MD3 purity was 99.4%, and impurities were negligible.

### Example 5: Thermal Decomposition Control Studies

Given MD3's chemical structure and potential for exothermic decomposition, a study was performed to fully evaluate its thermal decomposition properties and impact sensitivity. Specifically, the thermal decomposition properties of MD3 were characterized using differential scanning calorimetry. MD3 undergoes violent exothermic decomposition (detonation) in the solid state at 187° C. Likewise, the decomposition energy was determined to be 3754 J/g. As background, a substance is considered a candidate for classification as a United Nations Class I explosive if the heat of decomposition is ≥500 J/g. While a substance with a higher energy content is not necessarily highly sensitive or hazardous, the sensitivity of a high energy content material must be understood to ensure safe handling. Therefore, MD3's sensitivity to impact, friction forces, and electrostatic discharge was tested.

The threshold initiation limit (TIL₆) is the lowest energy level that does not cause the material to detonate when the test is replicated six times. The TIL₆ for solid MD3 was 7.5 J of impact energy. For reference, highly sensitive materials will decompose/detonate at energies of 2 J or less.

Notably, wetting MD3 with water at a 1 to 1 ratio, resulted in no decomposition up to 50 J of impact energy. Likewise, the TIL₆ for solid MD3 was 80 N of frictional force. When wetted with water at a 1 to 1 ratio, no decomposition of MD3 was observed up to 360 N.

Finally, the TIL₆ for solid MD3 was 63 mJ of electrostatic discharge (at 5 KV). When wetted with water at a 1 to 1 weight ratio, no decomposition of MD3 was observed up to 4.5 J (at 30 KV).

As MD3 was effectively desensitized to impact, frictional forces, and electrostatic discharge by dilution in an inert substance (water), control procedures were developed to process, store, handle, and ship MD3 as an aqueous solution. By blending with higher ratios of water, the energy content of the MD3 mixture/solution was diluted to the point where the mixture is no longer classified as an explosive or self-reactive material. For example, at a concentration of 64 mg/mL, the energy content of the solution remains below 300 J/g. As the final form of the MD3 drug substance is a solution, special control of the solid form is not required.

The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions, methods, and aspects of these compositions and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A method of manufacturing a high purity nitric oxide releasing compound product preparation, comprising:
preparing in a vessel a reaction mixture comprising a hydroxide or a hydroxide donor and one or more reaction mixture solvents;
introducing nitric oxide into the vessel under an inert atmosphere to obtain a suspension comprising a nitric oxide releasing compound;
filtering the suspension to obtain a nitric oxide releasing compound solid;
dissolving the nitric oxide releasing compound solid in an aqueous solution to form a dissolved nitric oxide releasing compound solution;
recrystallizing the nitric oxide releasing compound solid from the dissolved nitric oxide releasing compound solution to form a recrystallized nitric oxide releasing compound product, wherein the recrystallizing comprises a step of adding methanol to the dissolved nitric oxide releasing compound solution;
filtering the recrystallized nitric oxide releasing compound product and washing the filtered recrystallized nitric oxide releasing compound product with a mixture of washing solvents to form a washed nitric oxide releasing compound product, wherein the mixture of washing solvents comprises methanol and one or more additional alcohols;
drying the washed nitric oxide releasing compound product to form a dried nitric oxide releasing compound product; and
dissolving the dried nitric oxide releasing compound product in water to form the high purity nitric oxide releasing compound product preparation.

2. The method of claim 1, wherein the high purity nitric oxide releasing compound product preparation has a purity of at least 97 area % as measured by high pressure liquid chromatography.

3. (canceled)

4. The method of claim 1, wherein the high purity nitric oxide releasing compound product preparation has an impurity level of less than 1 area % as measured by high pressure liquid chromatography.

5. (canceled)

6. The method of claim 1, wherein the high purity nitric oxide releasing compound product preparation has a residual solvent level of less than 1.5% w/w.

7. (canceled)

8. The method of claim 1, wherein the reaction mixture comprises a hydroxide selected from the group consisting of sodium hydroxide, potassium hydroxide, lithium hydroxide, calcium hydroxide, magnesium hydroxide, and ammonium hydroxide, or
wherein the reaction mixture comprises a hydroxide donor selected from the group consisting of sodium methoxide, lithium methoxide, potassium methoxide, calcium methoxide, magnesium methoxide, and ammonium methoxide.

9. (canceled)

10. The method of claim 1, wherein the one or more reaction mixture solvents comprises an alcohol, a ketone, or both.

11. (canceled)

12. (canceled)

13. (canceled)

14. The method of claim 1, wherein the aqueous solution comprises water.

15. The method of claim 1, wherein the recrystallizing comprises adding a nitric oxide releasing compound seed during the step of adding methanol to the dissolved nitric oxide releasing compound solution.

16. The method of claim 15, wherein adding the nitric oxide releasing compound seed is initiated after a concentration of methanol in the dissolved nitric oxide releasing compound solution reaches at least 15% (v/v).

17. (canceled)

18. The method of claim 15, wherein the nitric oxide releasing compound seed is added to the dissolved nitric oxide releasing compound solution as a powder or as a suspension in a solvent.

19. (canceled)

20. (canceled)

21. The method of claim 1, wherein the mixture of washing solvents comprises methanol and a C₂-C₂₀ alcohol, wherein the C₂-C₂₀ alcohol is miscible with methanol.

22. (canceled)

23. The method of claim 21, wherein the mixture of washing solvents comprises a ratio of methanol to C₂-C₂₀ alcohol of 1:1 to 1:10.

24. (canceled)

25. The method of claim 1, wherein the dried nitric oxide releasing compound product is a crystalline form.

26. The method of claim 1, wherein the nitric oxide releasing compound comprises a compound having at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

27. The method of claim 26, wherein the compound has the following structure: 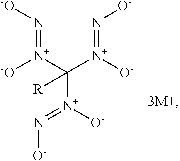
wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅ alkyl), —NH—((CH₂)_{d}NH)ₑ—H, and —NH—((CH₂)_{d}NH)ₑ—(CH₂)_{f}H;
a, b, c, d, e, and f are each independently selected from an integer of 0, 1, 2, 3, 4, 5, 6, 7, 8, 9, or 10; and
M+ is a pharmaceutically-acceptable cation, wherein a ratio of the compound to the cation is such that the overall net charge of the compound is neutral.

28. The method of claim 27, wherein the cation is selected from the group consisting of sodium, potassium, lithium, calcium, magnesium, ammonium, and substituted ammonium.

29. The method of claim 27, wherein the compound has the following structure:

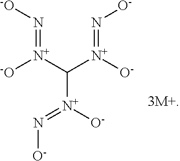

30. The method of claim 27, wherein the compound has the following structure:

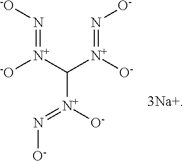

31. A high purity nitric oxide releasing compound product preparation prepared according to the method of claim 1.

32. (canceled)

33. A high purity nitric oxide releasing compound product, comprising:
a compound having at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation,
wherein the high purity nitric oxide releasing compound product has a purity of at least 97 area % as measured by high pressure liquid chromatography; and
wherein the high purity nitric oxide releasing compound product has a residual solvent level of less than 1.5% w/w.

34-42. (canceled)
